(19) United States
(12) Patent Application Publication
(10) Pub. No.: US 2005/0079544 A1
(43) Pub. Date: Apr. 14, 2005

(54) Automated method of metabolic stability analysis of library of compounds by isotope dilution mass spectrometry

(76) Inventors: Hoa Duc Nguyen, Orange, CA (US); Trinh Duc Nguyen, Anaheim, CA (US); Duc Tien Nguyen, Westminster, CA (US)

Correspondence Address: HIGH STANDARD PRODUCTS CORPORATION, SUITE 225, 14441 BEACH BLVD., WESTMINSTER, CA, 92683, US

(21) Appl. No.: 10/684,249

(22) Filed: Oct. 13, 2003

(51) Int. Cl. G01N 33/53; G01N 33/543

(52) U.S. Cl. 435/7.1; 436/518

(57) ABSTRACT

Method of automated metabolic stability analysis of library of compounds by mass spectrometry using stable isotope labeled internal standards is provided. Said internal standards are prepared in situ by reaction of an authentic sample of said library compounds with a stable isotope labeled reagent. In an automated fashion, cytochrome P450 enzyme systems are added to said library compounds; said reactions are terminated after certain periods of time by addition of an organic solvent containing equal amounts of said labeled internal standards; said terminated enzyme reactions are treated with a non-labeled version of said stable isotope labeled reagent to convert remaining library compounds to compounds of identical structures, except the labeled atoms, as those of said labeled internal standards; said conversion reactions are extracted and the extracts are analyzed by isotope dilution mass spectrometry to determine the percentage of library compounds remaining after enzyme reactions.

### BACKGROUND OF THE INVENTION

This invention pertains to method of quantitative analysis of library of organic compounds by isotope dilution mass spectrometry, particularly in a metabolic stability assay wherein said compounds are treated with enzymes of the liver to determine the extent of metabolism. Said compounds are usually drug candidates resulting from high throughput syntheses.

In searching for new drugs for a particular disease, scientists usually start from a lead compound and synthesize several hundreds of analogs for testing in a process called high throughput synthesis. These analogs, or chemical compounds with different chemical structure variations from the lead compound, constitute a so-called “library” of drug candidates. With careful synthesis planning, scientists can come up with a number of good candidates for the preliminary testing of efficacy. The good candidate compounds are then subjected to different assays with animal and human tissues to determine their safety as pharmaceuticals. Metabolic stability assay is an assay used to determine the extent of metabolism of a drug candidate compound when it passes through the liver. Said assay measures the remainder of a candidate compound as a percentage of its original concentration after its contact with the enzymes of the liver. The data from the assay will tell whether a drug candidate is extensively metabolized or poorly metabolized within a certain period of time in the body.

Metabolism is the mechanism by which the body uses to eliminate a drug substance. Because all body fluids are aqueous, said method of elimination, or metabolism, is the conversion of drugs into water soluble compounds. Enzymes in the liver are responsible for this conversion. Said enzymes of the liver are usually called the cytochrome P450 enzyme systems. Sources of enzymes include crude liver tissues, microsomes, S9 fractions, and individual isozymes. Said enzyme actions include hydroxylation, demethylation, ester hydrolysis, etc., wherein drugs are converted to the so-called “metabolites” which are drugs that are hydroxylated, demethylated, hydrolyzed, etc. Even though metabolites are no longer drugs, they are still structurally similar to drugs. Therefore, a reliable metabolic stability assay must include an analysis method that can positively identify and accurately quantitate a drug candidate among all of its structurally similar metabolites in the enzyme reaction medium. Mass spectrometric (MS) method is currently a method of choice for said analysis.

Said MS method identifies a drug candidate by its molecular weight ion and/or its daughter ion and measures its concentration by quantitation of these ions. To ease quantitation an internal standard is added to crude enzyme reactions after termination of reactions. Quantitation is made based on ratios of ions of drugs and said internal standard. Examples of metabolic stability assays using only one internal standard for the entire library of drug candidates are common. Usually no calibration curve is constructed and no extraction method for crude enzyme reactions is performed.

Analysis of crude enzyme reactions most often includes a centrifugation step to separate the macromolecules such as protein from the aqueous part. Without this protein separation step, analysis of the crude enzyme reactions will result in poor signal and build up solvent pressure. Automated centrifugation is difficult, but sample clean up by centrifugation is not all that desirable. If an extraction step is performed instead of a centrifugation step, the resulting mass spectrometric signal is usually much better. But sample clean up by extraction does have a disadvantage, that is, it is difficult to work out an extraction method wherein good recoveries of the internal standard and all drug candidates in the library are achieved. However, if stable isotope internal standard for each drug candidate is used, then good recoveries will not be necessary. The present invention discloses a metabolic stability assay using MS analysis which includes an automated synthesis of stable isotope labeled internal standards, an automated enzyme reaction protocol, an automated extraction method of crude enzyme reactions, and an MS analysis of said extracts.

A mass spectrometric analysis method using stable isotope labeled internal standard(s) is commonly called isotope dilution mass spectrometry. This method takes advantage of the similar chemical and physical behaviors of analytes and their respective isotope labeled internal standards towards all phases of sample preparation (extraction, derivatization, etc.) and also towards instrument responses (similar retention time, similar signal response, etc.). It uses the mass differentiation between analytes-and their respective internal standard for the quantitation of the ions. In a nutshell, a metabolic stability assay using isotope dilution MS analysis will provide accurate results regardless of the extraction recoveries of the drug candidate compounds and their labeled internal standards. All it requires is the availability of stable isotope labeled internal standard for each drug candidate compound.

The commonly used stable isotope labeled internal standard of an analyte is a chemical compound that has the same chemical structure as that of the analyte except that one or more substituent atoms are stable isotopes (deuterium, C-13, N-15, O-18). Synthesis of individual isotope labeled internal standards for said metabolic stability assay is an enormous task. The automated synthesis would be almost impossible. However, there are alternative ways to achieve the same objective. The objective is a short, reliable, and automated method of synthesis of stable isotope labeled internal standards that is suitable for the analysis of the compound library, but not the synthesis of stable isotope labeled drug candidates.

Isotope dilution mass spectrometric analysis requires that both analyte and its internal standard must have identical chemical structures, with the exception of the isotope atoms which provide the mass differentiation for quantitation. Said requirement ensures same extraction recoveries for both and same instrumental responses for both. If both said analyte and its stable isotope labeled internal standard are reacted with the same chemical reagent before analysis, then the products of said reactions must be structurally identical. This type of reaction is common in isotope dilution gas chromatography-mass spectrometry analysis (GC-MS) wherein the reagent used is usually called “derivatizing reagent”. The analysis becomes the analysis of the“derivatized” analyte and the “derivatized” internal standard, but their ion ratios remain unchanged. Automated metabolic stability assay using isotope dilution MS analysis method is feasible if both drug candidate and its internal standard are converted to compounds of identical structure, except the labeled atoms, before analysis. If said conversion is performed before extraction from the reaction medium, then their extraction recoveries will be the same. The present invention discloses said conversion in an automated fashion as a method to convert drug candidate compounds to compounds of identical structure as that of said isotope labeled internal standards in reaction medium before extraction wherein said labeled internal standards are already added.

Using well known efficient chemical conversion methods, stable isotope labeled internal standards are synthesized in an automated fashion from a portion of the drug candidate compounds and with only one stable isotope labeled chemical reagent. While the enzyme reactions of another portion of said drug candidate compounds are being carried out, said labeled internal standards are isolated by an automated extraction method and then added to appropriate enzyme reactions after they are terminated. A non-labeled version of said chemical reagent is added to said terminated enzyme reactions to transform said candidate compounds into compounds of identical structure, except the labeled atoms, as those of said added labeled internal standards. An automated extraction step for the crude enzyme reactions follows, and the final extracts are then analyzed by mass spectrometry.

Said efficient chemical conversion methods are selected based on the type of functional group present in said drug candidate compounds. For example, for said drug candidate compounds that have either a primary or a secondary amino group, said efficient conversion method is the transformation to an acetamide by using an excess of acetic acid anhydride. Said conversion is a one-step simple operation and can be performed in an automated fashion. One chemical reagent such as acetic acid anhydride-d6 can convert all the primary and secondary amino group containing drug candidate compounds in an automated fashion to acetamide-d6 analogs. The excess acetic anhydride-d6 is destroyed and said acetamide-d6 analogs are separated by an automated extraction and then added to enzyme reactions after they are terminated. Said terminated enzyme reactions are then, in an automated fashion, treated with excess acetic acid anhydride to transform said drug candidate compounds to acetamide analogs. Said acetamide-d6 analog in each terminated enzyme reaction is unreactive toward acetic acid anhydride. Both said acetamide-d6 analogs and said acetamide analogs in said terminated enzyme reactions are then extracted in an automated fashion, and the extracts are injected into a mass spectrometer for analysis.

This invention discloses an automated synthesis method for said drug candidate compounds that contain any of the four following functionality groups:
1. Group 1: a primary or a secondary amino functional group of a primary or a secondary amine compound.
2. Group 2: an hydroxyl functional group of an alcohol or a phenolic compound.
3. Group 3: a carbonyl functional group of an aldehyde or a ketone compound.
4. Group 4: a carboxyl functional group of a carboxylic acid compound.

Said efficient chemical conversion methods include:

For group 1, a primary or a secondary amino functional group:
1. conversion to an amide by using either an acid anhydride or an acid chloride.
2. conversion to a carbamate by using a chloroformate.
3. conversion to an urea by using an isocyanate.
4. conversion to a thiourea by using a thioisocyanate.

For group 2, a phenol or a hydroxyl functional group:
1. conversion to an ester by using either an acid anhydride or an acid chloride.
2. conversion to a carbamate by using an isocyanate.

For group 3, a carbonyl functional group of an aldehyde or a ketone:
1. conversion to an oxime by using an alkoxylamine.
2. conversion to a hydrazone by using an alkylhydrazine.

For group 4, a carboxyl functional group of a carboxylic acid: conversion to a carboxylic ester by using either a combination of alkylchloroformate and alcohol or a combination of base and alkyl halide.

Said efficient conversion reactions are selected based on the following criteria:
1. Said candidate compounds in said terminated enzyme reactions must be quantitatively converted to the compound of identical structure, except the labeled atoms, as that of said added isotope labeled internal standards using a non-labeled reagent.
2. Absolutely no conversion of said isotope labeled internal standard to said non-labeled analog because the conversion of said candidate compound occurs in said reaction medium in the presence of said added isotope labeled internal standard.
3. The conversion of said candidate compound into said compound of identical structure as that of said added isotope labeled internal standard has to be accomplished before extraction, not after extraction as in many cases of derivatization of GC-MS methods.

There are other conversion reactions that are very efficient, but said conversion reactions are very efficient in aqueous environment and can be performed at room temperature and in a relatively short reaction time. Said features are necessary for the invented automated metabolic stability assay using isotope dilution MS analysis.

### BRIEF SUMMARY OF THE INVENTION

The subject of the current invention provides for an automated method of metabolic stability analysis of a library of compounds. Said automated analysis include the following sequences:
1. automated synthesis and extraction of stable isotope labeled internal standards,
2. automated synthesis and extraction of non-label version of said internal standards for establishing parameters for mass spectrometric analysis,
3. automated reactions of cytochrome P450 enzyme systems with library compounds,
4. automated termination of said enzyme reactions and addition of said internal standards to said terminated reactions,
5. automated conversion of said library compounds in said enzyme reactions to compounds of identical structure as that of said internal standards,
6. automated extraction of said terminated enzyme reactions,
7. mass spectrometric analysis of said internal standards, said non-labeled version of said internal standards, and said enzyme reactions, and
8. calculation of percentage of remainder of library of compounds after enzyme reactions.

### DETAILED DESCRIPTION OF THE INVENTION

The method of the subject invention provides an automated metabolic stability assay and a quantitative analysis of library compounds after reactions with cytochrome P450 enzyme systems using isotope dilution mass spectrometric analysis. Chronologically, depending on the type of functional group present in said library compounds, the invention provides
methods of synthesis of stable isotope labeled internal standards of said library compounds using only one type of labeled chemical reagent,
methods of extraction of said internal standards,
methods of synthesis of a non-labeled version of said internal standards using the non-labeled version of said labeled chemical reagent,
methods of extraction of said non-labeled version of said internal standards for use in setting up parameters for mass spectrometric analysis,
methods of reactions of enzymes of the cytochrome P450 enzyme systems with library compounds,
methods of termination of enzyme reactions and addition of said extracts of said stable isotope labeled internal standards to said terminated enzyme reactions,
methods of conversion of remaining library compounds in said terminated enzyme reactions to compounds of identical structure as that of said internal standards using said non-labeled version of said labeled chemical reagent,
methods of extraction said internal standards and said converted compounds from said reaction medium,
methods of determination of molecular ion and product ion (also called daughter ion) of said internal standards and said converted compounds in said chemical synthesis extracts,
methods of selection of ion ratios of said molecular ions and said daughter ions and use of said ion ratios in MRM mode (multiple reaction monitoring mode),
methods of mass spectrometric analysis of said extracts of enzyme reactions in MRM mode,
methods of using mass spectrometric data of said selected ion ratios to determine percent remaining of said library compounds from said enzyme reactions.

Specifically, the method of the subject invention provides methods of synthesizing stable isotope labeled amide, carbamate, urea, and thiourea internal standards of primary and secondary amine compounds by reacting said compounds with a stable isotope labeled chemical reagents selected from a group consisting of labeled acid anhydrides or labeled acid chlorides, labeled chloroformates, labeled isocyanates, and labeled thioisocyanates. The invention provides methods of converting said amine compounds in said terminated enzyme reactions into compounds of identical structure as that of said labeled internal standards by adding to said terminated reactions a non-labeled version of said chemical reagents selected from a group consisting of non-labeled acid anhydrides or non-labeled acid chlorides, non-labeled chloroformates, non-labeled isocyanates, and non-labeled thioisocyanates.

Specifically, the method of the subject invention provides methods of synthesizing stable isotope labeled ester and carbamate internal standards of phenolic or alcoholic compounds by reacting said compounds with a stable isotope labeled chemical reagents selected from a group consisting of labeled acid anhydrides or labeled acid chlorides, and labeled isocyanates. The invention provides methods of converting said phenolic or alcoholic compounds in said terminated enzyme reactions into compounds of identical structure as that of said labeled internal standards by adding to said terminated reactions a non-labeled version of said chemical reagents selected from a group consisting of non-labeled acid anhydrides or non-labeled acid chlorides, and non-labeled isocyanates.

Specifically, the method of the subject invention provides methods of synthesizing stable isotope labeled oxime and hydrazone internal standards of aldehyde or ketone compounds by reacting said compounds with a stable isotope labeled chemical reagents selected from a group consisting of labeled alkoxylamines, and labeled alkylhydrazines. The invention provides methods of converting said aldehyde and ketone compounds in said terminated enzyme reactions into compounds of identical structure as that of said labeled internal standards by adding to said terminated reactions a non-labeled version-of said chemical reagents selected from a group consisting of non-labeled alkoxylamines and non-labeled alkylhydrazines.

Specifically, the method of the subject invention provides methods of synthesizing stable isotope labeled ester internal standards of carboxylic acid compounds by reacting said compounds with either a stable isotope labeled alcohol or a labeled alkyl halide. The invention provides methods of converting said carboxylic acid compounds in said terminated enzyme reactions into compounds of identical structure as that of said labeled internal standards by adding to said terminated reactions a non-labeled version of said labeled alcohol or said labeled alkyl halide.

Specifically, said cytochrome P450 enzyme systems in said metabolic stability assay can be cryopreserved or fresh human and animal hepatocytes, microsomes, S9 fractions or solutions containing individual cytochrome P450 isoenzymes.

Specifically, said enzyme reactions include reaction at time zero wherein reaction is terminated immediately after enzyme addition. Said MS data of said time zero reaction are used as original concentration of library compounds.

Specifically, said enzyme reactions include reactions at different time intervals wherein each reaction is terminated at specific time after enzyme addition. Said MS data of said time reactions are used to calculate the percentage of remaining of library compounds at said time intervals.

Specifically, the method of the subject invention provides methods of extraction of reactions by solid liquid extraction, liquid liquid extraction, and solid phase extraction.

### EXAMPLE

A library of 4 amines was analyzed for their metabolic stability using human microsomal proteins as the cytochrome P450 enzyme system. Aliquots of each amine were automatically placed in test tubes which were used for synthesis of its internal standard, synthesis of reference standards, and reactions with cytochrome P450 enzyme system. The internal standard of each amine is the acetamide-d3 compound formed by reaction of amines with acetic anhydride-d6. At the same time an aliquot of each amine was treated with acetic anhydride to form acetamide reference standard. Deuterated acetamide internal standards and acetamides were then separated by an automated extraction procedure. The reactions of amines with human microsomal protein were carried out in test tubes in 37° C. bath and was terminated by aspirating equal aliquots of the reaction solutions into test tubes containing equal volume of acetonitrile as terminating reagent. Reactions were aspirated at 0, 30, 60, 90, and 120 minutes. After all reactions were stopped, equal volumes of extracted acetamides-d3 were added to respective reactions as internal standards. Aqueous sodium bicarbonate and acetic anhydride were then added to convert remained amine in each reaction to the acetamide while leaving its acetamide-d3 internal standard unchanged. The tubes were mixed gently for 10 minutes. Both acetate amide and its acetate amide-d3 were separated from the reaction by an automatic extraction procedure. Solutions of extracts containing acetamide-d3, acetamide, and both from reactions with cytochrome P450 enzyme sytem were analyzed by electrospray mass spectrometry. Molecular ion and its product ion (daughter ion) of all acetamides and acetamides-d3 were determined in an automated fashion using an autosampler, a mass spectrometer and its analysis software. After all interested ions were determined, analysis of reaction extracts in MRM mode followed. The ion ratios of acetate amide to acetate amide-d3 were calculated and plotted against time.

Details of automated sequences are as follows:
1. Division of the individual amine into multiple equal amounts
   Via a standard liquid handler, aliquots of 4 amines (amine 1, 2, 3, and 4) were placed in 3 rows of 4 test tubes at volume of 0.005 ml and concentration of 1 mg/ml in methanol. Row 1 was for synthesis of internal standards, row 2 for synthesis of reference standards, and row 3 for reactions with human microsomal protein.
2. Synthesis of acetamide-d3 internal standards and acetamide reference standards
   Row 1 test tubes were treated with 0.1 ml of 10% v/v acetic anhydride-d6 in ethyl acetate while row 2 test tubes were treated with 0.1 ml of 10% v/v acetic anhydride in ethyl acetate. Next, aliquots of 0.1 ml 1M sodium bicarbonate were added to both rows 1 and 2. Tubes were mixed and allowed to stand for 10 minutes. Then, reactions were aspirated into filters containing hydromatrix® powder and aliquots of 0.900 ml ethyl acetate were added to filters. Filtrates contaning acetate amide-d3 were collected in new row 4 test tubes while filtrates contaning acetate amide were collected in new row 5 test tubes. Each of these filtrate solutions were assumed to be at the concentration of 0.005 mg/ml in ethyl acetate.
3. Reactions of amines with human microsomal protein
   Row 3 test tubes containing 0.005 ml of amine at 1 mg/ml in methanol were treated with 0.345 ml of 0.1M phosphate buffer pH 7 and 0.025 ml of 20 mg/ml human microsomal protein. The test tubes were incubated at 37° C. and were added 0.125 ml of buffer containing NADP (1.7 mg/ml), glucose-6-phosphate (7.8 mg/ml), glucose-6-phosphate dehydrogenase (1.5 units/ml), and sodium bicarbonate (20 mg/ml).
   Immediately at time 0, 0.100 ml of row 3 reactions were aspirated to row 6 test tubes containing 0.100 ml acetonitrile each. At time 30 minutes, 0.100 ml of row 3 reactions were aspirated to row 7 test tubes containing 0.100 ml acetonitrile each. At time 60 minutes, 0.100 ml of row 3 reactions were aspirated to row 8 test tubes containing 0.100 ml acetonitrile each. At time 90 minutes, 0.100 ml of row 3 reactions were aspirated to row 9 test tubes containing 0.100 ml acetonitrile each. At time 1200 minutes, 0.100 ml of row 3 reactions were aspirated to row 10 test tubes containing 0.100 ml acetonitrile each.
4. Sample processing of microsome reactions
   Rows 6,7,8,9, and 10 test tubes contained microsome reactions that was terminated by 0.100 ml acetonitrile. An aliquot of 0.050 ml solution of acetate amide-d3 in row 4 test tubes was aspirated to each of rows 6,7,8,9, and 10 test tubes. Next, an aliquot of 0.1 ml of 10% v/v acetic anhydride in ethyl acetate and an aliquot of 0.1 ml 1M sodium bicarbonate were added to rows 6,7,8,9, and 10 test tubes. The test tubes were gently shaked for 10 minutes and were aspirated into filters containing hydromatrix® powder that was sitting above test tubes of rows 11, 12, 13, 14, and 15. Aliquots of 1 ml ethyl acetate were aspirated into filters and filtrates were collected in rows 11, 12, 13, 14, and 15 test tubes.
5. Automated mass spectrometric analysis
   Aliquots of 0.050 ml of rows 4, 5, 11, 12, 13, 14, and 15 test tubes were placed in autosampler vials for mass spectrometric analysis. Total of 28 vials (1 to 28) were programmed for electrospray MS analysis using Q1 scan mode and product ion scan mode for reference standards and internal standards (vial 1 to 8), and using MRM mode for microsome reactions (vials 9 to 28). MRM mode of analysis or tandem MS analysis of 4 amines were set up as follows:
   Amine 1: acetamide 178.1→91.0; acetamide-d3: 181.2→91.0
   Amine 2: acetamide 192.2→91.0; acetamide-d3 195.2→91.0
   Amine 3: acetamide 236.2→105.0; acetamide-d3: 239.3→105.0
   Amine 2: acetamide 250.0→105.0; acetamide-d3: 253.2→105.0

Ion ratios of acetamide to acetamide-d3 for amine 1 to 4 are as follows:

| | 0 min | 30 min | 60 min | 90 min | 120 min |
|---|---|---|---|---|---|
| Amine 1 | 0.45 | 0.31 | 0.33 | 0.28 | 0.27 |
| Amine 2 | 0.35 | 0.22 | 0.21 | 0.23 | 0.19 |
| Amine 3 | 0.36 | 0.20 | 0.22 | 0.22 | 0.21 |
| Amine 4 | 0.92 | 0.60 | 0.69 | 0.80 | 0.72 |

6. Calculation of percent of remained amine compounds

Using the amount of amine at time t=0 as 100% , the % of remained amine with time is tabulated as follows:

| | T = 0 min | 30 min | 60 min | 90 min | 120 min |
|---|---|---|---|---|---|
| Amine 1 | 100% | 69% | 73% | 62% | 60% |
| Amine 2 | 100% | 63% | 60% | 65% | 54% |
| Amine 3 | 100% | 56% | 61% | 61% | 58% |
| Amine 4 | 100% | 65% | 75% | 87% | 78% |

References

US patent documents

| | | |
|---|---|---|
| 5,559,038 | Sep. 24, 1996 | J. Fred Kolhouse |
| 6,358,996 | Mar. 19, 2002 | Michael S. Alexander |

Other References

Arun K. Ghosh et al, “Stereoselective reduction of alpha-hydroxy oxime ethers: a convenient route to cis-1,2-amino alcohols”, Tetrahedron Letters, 1991, p.711-714, vol.32.

Barbara A. Way et al, “Isotope dilution GC-MS measurement of tricyclic antidepressant drugs. Utility of the 4-carbethoxyhexafluorobutyryl derivatives of secondary amines”, Journal of Analytical Toxicology, September 1998, page 374-382, vol. 22.

Dennis J. Dietzen et al, “Facilitation of thin-layer chromatographic identification of opiates by derivatization with acetic anhydride or methoxyamine”, Journal of Analytical Toxicology, September 1995, page 299-303, vol. 19.

Dong-Liang-Lin et al, “Chemical derivatization and the selection of deuterated internal standard for quantitative determination-methamphetamine example”, Journal of Analytical Toxicology, May/June 2000, page 275-280, vol. 24.

Hideyuki Yamada et al, “Dansyl chloride derivatization of methamphetamine: a method with advantages for screening and analysis of methamphetamine in urine”, Journal of Analytical Toxicology, January/February 2002, page 17-22, vol. 19.

Hiroshi Goda et al, “Facile synthesis of 5-substituted 2-acetylthiophenes”, Synthesis, 1992, p.849-851.

J. G. Guillot et al, “Determination of heroin, 6-acetylmorphine, and morphine in biological fluids using their propionyl derivatives with ion trap GC-MS”, Journal of Analytical Toxicology, March/April 1997, page 127-133, vol. 21.

Jens Pietzsch et al, “Rapid determination of total homocysteine in human plasma by using N(O,S)-Ethoxycarbonyl ethyl ester derivatives and gas chromatography-mass spectrometry”, Clinical Chemistry, 1997, page 2001-2004, vol. 43(10).

Kenji Hara et al, “Simple extractive derivatization of methamphetamine and its metabolites in biological materials with extrelut columns for their GC-MS determination”, Journal of Analytical Toxicology, January/February 1997, page 54-58, vol. 21.

Kyle R. Gee et al, “Arene chromium and manganese tricarbonyl analogs of the PCP receptor ligands 5-methyl-10,11 -dihydro-5-H-dibenzo[a,d]cyclohepten-5,10-imine (MK-801) and 10,5-(iminomethano)-10-11-dihydro-5H-dibenzo[a,d]cycloheptene” Journal of Organic Chemistry, 1994, p. 1492-1498, vol.59.

Maciej Bogusz et al, “Analysis of underivatized amphetamines and related phenethylamines with HPLC-APCI-MS”, Journal of Analytical Toxicology, March 2000 page 77-84, vol. 24.

Maciej Bogusz, “Determination of phenylisothicyanate derivatives of amphetamine and its analogues in biological fluids by HPLC-APCI-MS or DAD”, Journal of Analytical Toxicology, January/February 1997, page 59-69, vol. 21.

Mark M. Kushnir et al, “Comparison of four derivatizing reagents for 6-acetylmorphine GC-MS analysis”, Journal of Analytical Toxicology, July/August 1999, page 262-269, vol. 23.

Michael L. Smith et al, “Forensic drug testing for opiates. VI. Urine testing for hydromorphone, hydrocodone, oxymorphone, and oxycodone with commercial opiate immunoassay and GC-MS”, Journal of Analytical Toxicology, January/February 1995, page 18-26, vol. 19.

Nieves Pizarro et al, “Determination of MDMA and its metabolites in blood and urine by GC-MS and analysis of enantiomers by capillary electrophoresis”, Journal of Analytical Toxicology, April 2002, page 157-165, vol. 26.

P. Dallakian et al, “Detection and quantitation of amphetamine and methamphetamine: El and CI with ammonia—comparative investigation on Shimadzu QP5000 GC-MS system”, Journal of Analytical Toxicology, July/August 1996, page 255-261, vol. 20.

Petr Husek and Petr Simek, “Advances in amino acid analysis”, LCGC September 2001, page 986-999, vol.19.

Petr Husek, “Chloroformates in gas chromatography as general purpose derivatizing agents”, Journal of Chromatography B, 1998, page 57-91, vol. 717.

Ping Cao and Mehdi Moini, “Quantitative analysis of fluorinated ethylchloroformate derivatives of protein amino acids and hydrolysis products of small peptides using chemical ionization gas chromatography-mass spectrometry”, Journal of Chromatography A, 1997, page 111-117, vol. 759.

Robert Meatherall, “Rapid GC-MS confirmation of urinary amphetamine and methamphetamine as their propylchloroformate derivatives”, Journal of Analytical Toxicology, September 1995, page 316-322, vol. 19.

William A. Joern, “Unexpected volatility of barbiturate derivatives : an extractive alkylation procedure for barbiturates and benzoylecgonine”, Journal of Analytical Toxicology, November 1994 page 423, vol. 18.

1. An automated method of determination of metabolic stability of library of compounds from cytochrome P450 enzyme reactions by isotope dilution mass spectrometric analysis comprising the steps of:
a) converting a portion of each said library compound into a stable isotope labeled internal standard by addition of a stable isotope labeled chemical reagent; and
b) converting another portion of each said library compound into a compound of identical structure, with the exception of the stable isotope atoms, as that of said stable isotope labeled internal standard comprising addition of a non-isotope version of said stable isotope reagent; and
c) separating said stable isotope labeled internal standards and said non-isotope labeled converted compounds by an extraction method; and
d) incubating other portions of said library of compounds in the presence of cytochrome P450 enzyme systems at body temperature for periods of time; and
e) adding quenching chemical reagents to terminate said enzyme reactions; and
f) adding said stable isotope labeled internal standards to said terminated reactions; and
g) adding said non-isotope version of said stable isotope labeled chemical reagent to said terminated reactions to convert the remaining of each said library compound into said non-isotope labeled converted compounds; and
h) separating said converted compound and its internal standard from said reactions by an extraction method; and
i) determining the molecular ions of said stable isotope labeled internal standards and said non-isotope labeled converted compounds; and
j) determining the most abundant daughter ions of said stable isotope labeled internal standards and said non-isotope labeled converted compounds; and
k) determining the ion ratio of said converted compound to said corresponding internal standard for each of said enzyme reactions using tandem mode of mass spetrometric analysis; and
l) determining the percent remaining of each said library compound from said enzyme reactions from said ion ratios.

2. The method of claim 1 wherein said steps a), b), and d) are performed concurrently.

3. The method of claim 1 wherein said extraction in steps c) and h) can be any appropriate separating methods such as solid phase extraction, liquid-liquid extraction or solid supported liquid-liquid extraction.

4. The method of claim 1 wherein said sample contains either a singularity or a plurality of each class of said library compounds.

5. The method of claim 1 wherein said library compounds in step a) are converted to said internal standards using a single isotope labeled chemical reagent.

6. The method of claim 1 wherein said library compounds in steps b) and g) are converted to compounds of identical structure as that of said internal standards, except the labeled atoms, using a single non-labeled version of said chemical reagent.

7. The method of claim 1 wherein said conversion in steps a), b) and g) are 100% quantitative.

8. The method of claim 1 wherein the converting step g) is performed before the extraction step h).

9. The method of claim 1 wherein said quenching chemical reagent is an organic solvent.

10. The method of claim 1 wherein said library compounds are all primary and/or secondary amines and said stable isotope reagent is selected from a group consisting of a labeled acid anhydride or labeled acid chloride, labeled chloroformate, labeled isocyanate, and labeled thioisocyanate, and said resulting internal standards are labeled amides, labeled carbamates, labeled ureas, and labeled thioureas, respectively.

11. The method of claim 1 in which said library compounds are all alcohols and/or phenols and said stable isotope reagent is selected from a group consisting of an labeled acid anhydride or labeled acid chloride and labeled isocyanate, and said resulting internal standards are isotope labeled esters and isotope labeled carbamates, respectively.

12. The method of claim 1 in which said library compounds are all aldehydes and/or ketones and said stable isotope reagent is selected from a group consisting of a labeled alkoxylamine and a labeled alkylhydrazine, and said resulting internal standards are labeled oximes and labeled hydrazones, respectively.

13. The method of claim 1 in which said library compounds are all carboxylic acids and said stable isotope reagent is either a labeled alcohol and a chloroformate or a labeled alkyl halide and a base and said resulting internal standards are labeled carboxylic acid esters.

14. The method of claim 1 wherein said cytochrome P450 enzyme systems are cryopreserved or fresh human and animal hepatocytes, microsomes, S9 fractions or solutions containing individual cytochrome P450 isoenzymes.

15. The method of claim 1 wherein said library compounds are all primary or secondary amines and said non-labeled version of said stable isotope labeled reagent in steps b and g is selected from a group consisting of an acid anhydride or acid chloride, a chloroformate, an isocyanate, and a thioisocyanate and said resulting converted compounds are amides, carbamates, ureas and thioureas, respectively.

16. The method of claim 1 wherein said library compounds are all alcohols and/or phenols and said non-labeled version of said stable isotope labeled reagent in steps b and g is selected from a group consisting of an acid anhydride or acid chloride and an isocyanate, and said resulting converted compounds are esters and carbamates, respectively.

17. The method of claim 1 wherein said library compounds are all aldehydes and/or ketones and said non-labeled version of said stable isotope labeled reagent in steps b and g is selected from a group consisting of an alkoxylamine and an alkylhydrazine, and said resulting converted compounds are oximes and hydrazones, respectively.

18. The method of claim 1 wherein said library compounds are all carboxylic acids and said non-labeled version of said stable isotope labeled reagent in steps b and g is either an alcohol and a chloroformate or an alkyl halide and a base, and said resulting converted compounds are esters.
